(19) United States
(12) Patent Application Publication
(10) Pub. No.: US 2025/0109409 A1
(43) Pub. Date: Apr. 3, 2025

(54) RIG1 Inhibition and Therapeutic Viral Protein Expression

(71) Applicant: ImmunityBio, Inc., Culver City, CA (US)

(72) Inventor: Patrick Soon-Shiong, Culver City, CA (US)

(73) Assignee: ImmunityBio, Inc., Culver City, CA (US)

(21) Appl. No.: 18/823,196

(22) Filed: Sep. 3, 2024

Related U.S. Application Data
(60) Provisional application No. 63/587,008, filed on Sep. 29, 2023.

(51) Int. Cl. C12N 15/86; A61K 38/17; A61K 38/20; C07K 14/005; C12N 15/113

(52) U.S. Cl. CPC C12N 15/86; A61K 38/1793; A61K 38/2086; C07K 14/005; C12N 15/113; C12N 2310/11; C12N 2310/141; C12N 2710/10043; C12N 2830/008

(57) ABSTRACT

Viral transfection and expression of recombinant viral payload in a transfected cell is enhanced by co-expression of a means of interfering with the expression of Retinoic Acid-Inducible Gene I (RIG-1). In some embodiments, the co-expression is driven from a tissue specific promoter.

This application claims priority to our copending US Provisional Patent application with the Ser. No. 63/587,008, which was filed Sep. 29, 2023, and which is incorporated by reference herein.

### FIELD OF THE INVENTION

The field of the invention is compositions and methods for transfecting cells, especially as it relates to transfection of muscle cells with a recombinant nucleic acid that encodes a viral antigenic peptide and a means of interfering with the expression of Retinoic Acid-Inducible Gene I (RIG-1).

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Recombinant expression of a large variety of proteins in mammalian cells is routinely practiced in the pharmaceutical and therapeutic arts. While conceptually relatively simple, there are numerous difficulties associated with recombinant expression. Among other things, proper folding of the recombinant protein in the transfected cells can be challenging, which may be further compounded by differences in glycosylation patterns. Moreover, and depending on the particular cell type and location to be transfected, the number of transfection methods will narrow rapidly, especially where the cells are cells in vivo. In many instances, viral transfection is often a preferred route as such route can be taken in vitro and in vivo. For example, there are a number of therapeutic viruses such as adenoviruses, adeno-associated viruses, vaccinia viruses, herpesviruses, coxsackievirus, etc.

However, the rate of transfections with therapeutic viruses is often less than desirable, even when a relatively high multiplicity of infection (MOI) is used. Among other factors, selective metabolic pressure on infected cells may lead to disfavored viral replication and transcription/translation. Additionally, many therapeutic viruses can trigger an immune response in an infected subject, rendering subsequent infections with the therapeutic virus often ineffective. To circumvent such difficulties, the recombinant viral genome can be modified by removing antigenic proteins as was, for example, reported with the adenovirus serotype 5 (see e.g., J Virol. 1998 February; 72(2):926-33). Here, the deletion of the E1 and E2b genes resulted in significantly decreased immunogenicity and increased stabilization of the viral genome. Unfortunately, such modified viruses still suffered from decreased vector gene expression (see e.g., Clin Vaccine Immunol. 2017 Jun. 5; 24(6):e00061-17).

Thus, even though various compositions and methods of recombinant expression of proteins using viral expression systems are known in the art, all or almost all of them suffer from several drawbacks. Therefore, there remains a need for improved recombinant viral expression systems.

### SUMMARY OF THE INVENTION

The inventive subject matter is directed to various compositions and methods of transfecting a cell, and especially a muscle cell, with a recombinant nucleic acid that includes a segment comprising a muscle-tissue specific promoter that is operationally coupled to a nucleic acid sequence encoding a means of interfering with the expression of Retinoic Acid-Inducible Gene I. Consequently, so transfected cells lack or have a reduced capability to degrade viral RNA, leading to increased recombinant protein production as compared to cells not transfected with such recombinant nucleic acid.

In one aspect of the inventive subject matter, the inventor contemplates a method of expressing viral RNA in a muscle cell that includes a step of transfecting into the muscle cell a nucleic acid vector comprising a muscle-tissue specific promoter and a nucleic acid sequence encoding a means of interfering with the expression of Retinoic Acid-Inducible Gene I (RIG-I), and a step of transfecting into the muscle cell a viral vector comprising a nucleic acid sequence that encodes at least one viral antigenic peptide.

In some embodiments, the viral vector comprises an adenoviral vector (e.g., AdV vector with at least an E2b deletion), and in further embodiments the nucleic acid vector comprises a viral vector or an RNA vector. Most typically, the expression of type I interferon (IFN) is reduced, compared to the expression of type I IFN in a muscle cell transfected with the second nucleic acid vector alone. Additionally, it is contemplated that a peptide sequence encoded by the viral nucleic acid is expressed and presented by a major histocompatibility complex protein on the muscle cell surface, but not expressed and presented by a muscle cell transfected with the second nucleic acid vector alone.

For example, contemplated means of interfering with the expression of RIG-I comprises a dsRNA, a siRNA, a shRNA, a IncRNA or means of silencing thereof, or a miRNA (e.g., miR-485), nucleic acid binding site thereof, or means of silencing thereof. In further contemplated examples, the muscle-tissue specific promoter comprises an α-actin promoter, a muscle creatine kinase promoter, or a desmin gene promoter. Alternatively, the muscle-tissue specific promoter may comprise a human α-myosin heavy chain gene (αMHC) promoter, a myosin light-chain promoter, or a cardiac troponin T promoter, or the muscle-tissue specific promoter may comprise a synthetic promoter. As will be readily appreciated, the nucleic acid vector and/or the viral vector further comprises an enhancer region.

Consequently, the inventor also contemplates a method of expressing viral RNA in a muscle cell, the method comprising transfecting into the muscle cell a viral vector comprising 1) a muscle-tissue specific promoter, 2) a nucleic acid sequence comprising a means of interfering with the expression of Retinoic Acid-Inducible Gene I (RIG-I), and 3) a nucleic acid sequence encoding an antigenic viral peptide; wherein the promoter drives expression of the means of RIG-I interference.

Therefore, and viewed from a different perspective, the inventor also contemplates a composition that comprises at least one viral vector comprising a muscle-tissue specific promoter, a nucleic acid sequence encoding a means of interfering with the expression of Retinoic Acid-Inducible Gene I (RIG-I), and a nucleic acid sequence encoding a viral peptide. Preferably, but not necessarily, the at least one viral vector comprises an adenoviral vector, or an RNA viral vector. As noted above, it is contemplated that the means for interfering with expression of RIG-1 comprises a dsRNA, a siRNA, a shRNA, a IncRNA or means of silencing thereof, or a miRNA, nucleic acid binding site thereof, or means of silencing thereof. Alternatively, or additionally, the means of interfering may also comprise an antisense DNA, or a genome editing component such as a CRISPR-Cas9, a TALEN, or a Zinc finger nuclease.

In typical embodiments, the muscle-tissue specific promoter comprises an α-actin promoter, a muscle creatine kinase promoter, a desmin gene promoter, a human α-myosin heavy chain gene (αMHC) promoter, a myosin light-chain promoter, a cardiac troponin T promoter, or a synthetic promoter.

Thus, in still another aspect of the inventive subject matter, the inventor also contemplates a method of stimulating an immune response in a patient in need thereof that includes a step of administering the composition presented herein to a patient in need thereof. Of course, contemplated compositions may further include immunostimulant or an immune stimulant is co-administered. For example, especially contemplated immunostimulants comprise IL-15 or an agonist derivative thereof (e.g., N-803).

Various objects, features, aspects, and advantages of the inventive subject matter will become more apparent from the following detailed description of preferred embodiments.

### DETAILED DESCRIPTION

RIG-I (retinoic acid-inducible gene I) is a pattern recognition receptor in the cytosol of mammalian cells that binds single and double stranded viral RNA and mediates the induction of a type-I interferon (IFN1) response. As such, RIG-I is a critical component in the innate immune system that recognizes viral infection in a cell, leading to expression and presentation of viral proteins on the MHC complex and ultimately the destruction of the infected cell.

The inventor has now discovered that many recombinant viral expression vectors and recombinant viruses (e.g., adenoviral expression vectors) will trigger a RIG-1 response due to the presence of the recombinant viral RNA in a transfected cell. Consequently, the recombinant payload is rapidly destroyed and not expressed in meaningful quantities, and cells infected with the recombinant virus are eliminated from a subject. Such issues are further compounded where viral infection also includes peripheral blood mononuclear cells, and especially dendritic cells and macrophages, as such infection further enhances an immune response against the recombinant virus.

To circumvent, or at least reduce issues associated with a RIG-1 response in cells infected with a recombinant (typically therapeutic) virus, the inventor contemplates that the cells will be co-transfected (contemporaneously or serially) with both a RIG-1 knock-down/out system and a viral payload. As such, RIG-1 activity is specifically reduced in the infected cell, leading to higher levels of recombinant and/or viral proteins. With regard to suitable RIG-1 sequences in conjunction with the teachings presented herein, it should be understood that the knock-down/out system will be tailored to the specific source (and even isoform where expressed). Therefore, where the transfected cells are human cells, a knock-down/out system for the human form of RIG-1 (see NIH Gene ID: 23586) is contemplated. On the other hand, where the transfected cells are non-human cells (e.g., rodent, porcine, bovine, etc.), a knock-down/out system for the corresponding non-human homologs are particularly contemplated.

For example, in one exemplary embodiment, suitable recombinant viruses include various recombinant adenoviruses, and especially replication defective adenoviruses with E2b deletions such as described in U.S. Pat. Nos. 11,779,637, 11,071,774, and 11,590,217. However, it should be noted that numerous other (therapeutic) recombinant viral expression systems are also deemed suitable for use herein so long as such viruses are capable of leading to expression of the recombinant payload in an infected cell. For example, suitable viruses include genetically modified alphaviruses, adenoviruses, adeno-associated viruses, herpes viruses, lentiviruses, etc. In still further examples, genetically modified adenoviruses are preferred that are suitable not only for multiple vaccinations but also vaccinations in individuals with preexisting immunity to the adenovirus (see e.g., WO 2009/006479 and WO 2014/031178), typically achieved by deletion of the E2b gene and other late proteins to reduce immunogenicity. Moreover, due to these specific deletions, such genetically modified viruses were replication deficient and allowed for relatively large recombinant cargo. For example, WO 2014/031178 describes the use of such genetically modified viruses to express CEA (colorectal embryonic antigen) to provide an immune reaction against colon cancer.

Regardless of the type of recombinant virus it is contemplated that the virus may be used to infect patient (or non-patient) cells ex vivo or in vivo. For example, the virus may be injected intramuscularly, intratumorally, subcutaneously, or intravenously, or may be administered intranasally or via inhalation to so infect the patient's cells, and especially muscle cells and antigen presenting cells. Alternatively, cells of the patient (or from an allogeneic source) may be infected in vitro and then transfused to the patient. Of course, it should be recognized that immune therapy need not rely on virus particles but may also be effected by nucleic acid transfection using RNA or DNA.

As will be readily appreciated, contemplated recombinant viruses may include a nucleic acid sequence that encodes one or more viral and/or disease specific antigens, or recombinant proteins of interest, and optionally one or more functional molecules such as immune stimulatory molecules (e.g., cytokines, co-stimulatory molecules, checkpoint inhibitors, etc.), markers for quantification, etc. Therefore, suitable recombinant viruses may be therapeutic viruses that can be general therapeutics or recombinant viruses for personalized immune therapy.

As will further be appreciated, in at least some embodiments the recombinant virus may further comprise a nucleic acid that comprises a muscle (or other)-tissue specific promoter and a nucleic acid sequence encoding a means of interfering with the expression of Retinoic Acid-Inducible Gene I (RIG-I). Consequently, it should be recognized that infection of a target cell will result in expression of the recombinant viral nucleic acid, and with that produce within the cell a means of interfering with the expression of Retinoic Acid-Inducible Gene I (RIG-I). Due to the tissue specific promoter, it should further be noted that the RIG-1 dependent innate immunity is only reduced or abrogated in the specific targeted tissues. Consequently, it should be noted that numerous promoters can be used such as an α-actin promoter, a muscle creatine kinase promoter, a desmin gene promoter, human α-myosin heavy chain gene (αMHC) promoter, a myosin light-chain promoter, or a cardiac troponin T promoter, or even entirely synthetic promoter. Likewise, it should be appreciated that while muscle-tissue specific promoters are elaborated above, alternative tissue-specific promoters are also contemplated for use herein (see e.g., Molecular Systems Biology (2007)3:73). In less preferred aspects, promoters with promiscuous tissue specificity and even non-specific promoters are also contemplated.

Regardless of the particular cell type and promoter used, it should be appreciated that all known manners of reducing or abrogating RIG-1 expression are deemed suitable, and especially those manners that are under the control of the tissue specific promoter. For example, suitable means of interfering with the expression of IRG-1 include interference by dsRNA, siRNA, shRNA, IncRNA or means of silencing thereof, and/or miRNA (e.g., using miR-485), nucleic acid binding site thereof, or means of silencing thereof. Suitable DNA-based methods especially include antisense DNA silencing. The PHOSITA is well apprised of such gene silencing or gene knock-downs, and all of the known methods are deemed suitable for use herein. Alternatively, the RIG-1 gene may also be entirely eliminated (knocked-out), for example, using known genome editing tools based on CRISPR-Cas9, TALEN, or Zinc finger nucleases. Therefore, it should be recognized that (preferably tissue-specific) interference with RIG-1 will mitigate the induction of an immune response to a therapeutic viral nucleic acid. As a consequence, it is contemplated that in vivo expression of type I interferons (IFN) will be reduced, compared to the expression of type I IFNs in a muscle cell transfected with the second nucleic acid vector alone. Likewise, it should be appreciated that the peptide sequence encoded by the viral nucleic acid is expressed and presented by a major histocompatibility complex protein on the muscle cell surface, but not expressed and not presented by a muscle cell transfected with the second nucleic acid vector alone.

Therefore, the inventor also contemplates a method of expressing viral RNA in a muscle cell that includes a step of transfecting into the muscle cell a viral vector comprising 1) a muscle-tissue specific promoter, 2) a nucleic acid sequence comprising a means of interfering with the expression of RIG-1, and 3) a nucleic acid sequence encoding an antigenic viral peptide; wherein the promoter drives expression of the means of RIG-I interference. In view of such methods, the inventor also contemplates various compositions that comprise at least one viral vector comprising a muscle-tissue specific promoter, a nucleic acid sequence encoding a means of interfering with the expression of RIG-1, and a nucleic acid sequence encoding a viral peptide.

Using such compositions and methods in vivo, it should be appreciated that the inventor also contemplates a method of stimulating an immune response in a patient in need thereof, wherein the compositions presented herein are administered to the patient in need thereof. Where desired, such in vivo use may be further augmented by (co-)administering an immune stimulant. Among other suitable options, it is particularly preferred that the immune stimulant comprises IL-15 or an agonist derivative thereof (e.g., N-803).

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A method of expressing viral RNA in a muscle cell, the method comprising:
a. transfecting into the muscle cell a nucleic acid vector comprising a muscle-tissue specific promoter and a nucleic acid sequence encoding a means of interfering with the expression of Retinoic Acid-Inducible Gene I (RIG-I);
b. transfecting into the muscle cell a viral vector comprising a nucleic acid sequence that encodes at least one viral antigenic peptide.

2. The method of claim 1, wherein the viral vector comprises an adenoviral vector.

3. The method of claim 1, wherein the nucleic acid vector comprises a viral vector or an RNA vector.

4. The method of claim 1, wherein the expression of type I interferon (IFN) is reduced, compared to the expression of type I IFN in a muscle cell transfected with the second nucleic acid vector alone.

5. The method of claim 1, wherein a peptide sequence encoded by the viral nucleic acid is expressed and presented by a major histocompatibility complex protein on the muscle cell surface, but not expressed and not presented by a muscle cell transfected with the second nucleic acid vector alone.

6. The method of claim 1, wherein the means of interfering with the expression of RIG-I comprises a dsRNA, a siRNA, a shRNA, a IncRNA or means of silencing thereof, or a miRNA, nucleic acid binding site thereof, or means of silencing thereof.

7. The method of claim 6, wherein the miRNA comprises miR-485.

8. The method of claim 1, wherein the muscle-tissue specific promoter comprises an α-actin promoter, a muscle creatine kinase promoter, a desmin gene promoter, a human α-myosin heavy chain gene (αMHC) promoter, a myosin light-chain promoter, or a cardiac troponin T promoter.

9. The method of claim 1, wherein the muscle-tissue specific promoter comprises a synthetic promoter.

10. The method of claim 1, wherein the nucleic acid vector and/or the viral vector further comprises an enhancer region.

11. A method of expressing viral RNA in a muscle cell, the method comprising transfecting into the muscle cell a viral vector comprising 1) a muscle-tissue specific promoter, 2) a nucleic acid sequence comprising a means of interfering with the expression of Retinoic Acid-Inducible Gene I (RIG-I), and 3) a nucleic acid sequence encoding an antigenic viral peptide; wherein the promoter drives expression of the means of RIG-I interference.

12. A composition comprising at least one viral vector comprising a muscle-tissue specific promoter, a nucleic acid sequence encoding a means of interfering with the expression of Retinoic Acid-Inducible Gene I (RIG-I), and a nucleic acid sequence encoding a viral peptide.

13. The composition of claim 12, wherein the at least one viral vector comprises an adenoviral vector.

14. The composition of claim 12, wherein the at least one nucleic acid vector comprises an RNA viral vector.

15. The composition of claim 12, wherein the means for interfering with expression of RIG-1 comprises a dsRNA, a siRNA, an shRNA, a IncRNA or means of silencing thereof, or a miRNA, nucleic acid binding site thereof, or means of silencing thereof.

16. The composition of claim 12, wherein the means of interfering comprises antisense DNA.

17. The composition of claim 12, wherein the means of interfering comprises genome editing, wherein the genome editing comprises CRISPR-Cas9, TALEN, or Zinc finger nucleases.

18. The composition of claim 12, wherein the muscle-tissue specific promoter comprises an α-actin promoter, a muscle creatine kinase promoter, a desmin gene promoter, a human α-myosin heavy chain gene (αMHC) promoter, a myosin light-chain promoter, or a cardiac troponin T promoter.

19. The composition of claim 12, wherein the muscle-tissue specific promoter comprises a synthetic promoter.

20. A method of stimulating an immune response in a patient in need thereof, the method comprising administering the composition of claim 12 to a patient in need thereof, and optionally further comprising administering an immunostimulant selected from the group consisting of IL-15, an IL-15 agonist, and N-803.
